SEQUENCE LISTING (38 sequences)

SEQ ID NO: 1 - DNA, 2070 bp, Homo sapiens
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaaaaaaaaaaaaaaaaaa

SEQ ID NO: 2 - protein, 501 aa, Homo sapiens
  mat_peptide (22)..(501)
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 3 - DNA, 1804 bp, Homo sapiens
atgggcgcactggcccgggcgctgctgctgcctctgctggcccagtggctcctgcgcgccgccccggagctggcccccgcgcccttcacgctgcccctccgggtggccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagatgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatgaatagccaggcctgacctcaagcaaccatgaactcagctattaagaaaatcacatttccagggcagcagccgggatcgatggtggcgctttctcctgtgcccacccgtcttcaatctctgttctgctcccagatgccttctagattcactgtcttttgattcttgattttcaagctttcaaatcctccctacttccaagaaaaataattaaaaaaaaaacttcattctaaaccaaaaaaaaaaaaaaa

SEQ ID NO: 4 - protein, 518 aa, Homo sapiens
  mat_peptide (23)..(518)
MGALARALLLPLLAQWLLRAAPELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 5 - protein, 17 aa, Homo sapiens
ERHADGLALALEPAWKK

SEQ ID NO: 6 - protein, 56 aa, Homo sapiens
EISEVKMDAEFRHDSGYEVHHQKLVFFAEDVGSNKGAIIGLMVGGVVIATVIVITL

SEQ ID NO: 7 - protein, 12 aa, Artificial Sequence
  Synthetic peptide
MASMTGGQQMGR

SEQ ID NO: 8 - protein, 468 aa, Homo sapiens
  mat_peptide (16)..(468)
MASMTGGQQMGRGSMELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWHHHHHH

SEQ ID NO: 9 - protein, 415 aa, Homo sapiens
  mat_peptide (15)..(415)
MASMTGGQQMGRGSANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWHHHHHH

SEQ ID NO: 10 - protein, 407 aa, Homo sapiens
  mat_peptide (13)..(407)
MRGSHHHHHHGSANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILW

SEQ ID NO: 11 - protein, 447 aa, Homo sapiens
  mat_peptide (36)..(447)
MRGSHHHHHHGSIETDTQHGIRLPLRSGLGGAPLGDGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILW

SEQ ID NO: 12 - protein, 424 aa, Homo sapiens
  mat_peptide (13)..(424)
MRGSHHHHHHGSDGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILW

SEQ ID NO: 13 - DNA, 22 bp, Artificial Sequence
  Synthetic primer
ggcgtatcacgaggccctttcg

SEQ ID NO: 14 - DNA, 59 bp, Artificial sequence
  Synthetic primer
catggaactggctccagctccgttcactctgccactgcgtgttgctgcagctactaacc

SEQ ID NO: 15 - DNA, 58 bp, Artificial sequence
  Synthetic primer
gtgttgtagctccaactccaggaccaggtactccagctgaacgtcatgctgatggtct

SEQ ID NO: 16 - DNA, 57 bp, Artificial Sequence
  Synthetic primer
ggctctggctctggaaccagctctggcttctccagctggtgctgctaacttcctggc

SEQ ID NO: 17 - DNA, 56 bp, Artificial sequence
  Synthetic primer
catggccaggaagttagcagcaccagctggagaagccagagctggttccagagcca

SEQ ID NO: 18 - DNA, 57 bp, Artificial sequence
  Synthetic primer
gagccagaccatcagcatgacgttcagctggagtacctggtcctggagttggagcta

SEQ ID NO: 19 - DNA, 60 bp, Artificial Sequence
  Synthetic primer
caacacggttagtagctgcagcaacacgcagtggcagagtgaacggagctggagccagtt

SEQ ID NO: 20 - DNA, 37 bp, Artificial Sequence
  Synthetic primer
ggaagcttaatggtgatggtgatggtgccaaaagggc

SEQ ID NO: 21 - DNA, 34 bp, Artificial Sequence
  Synthetic primer
ctgcaggttgtctaccatagccaggaacttgcag

SEQ ID NO: 22 - DNA, 31 bp, Artificial Sequence
  Synthetic primer
ggtcaggatccaacaggagctcaagtcagct

SEQ ID NO: 23 - DNA, 32 bp, Artificial Sequence
  Synthetic primer
aagcttatcaccacaaaatgggctcgctcaag

SEQ ID NO: 24 - DNA, 28 bp, Artificial Sequence
  Synthetic primer
gctcggatccgctaacttcctggctatg

SEQ ID NO: 25 - DNA, 31 bp, Artificial Sequence
  Synthetic primer
gctcggatccgatggtctggctctggctctg

SEQ ID NO: 26 - DNA, 31 bp, Artificial Sequence
  Synthetic primer
tccactgggtgatggtctggctctggctctg

SEQ ID NO: 27 - DNA, 31 bp, Artificial Sequence
  Synthetic primer
ccagaccatcaggcagtggagcaccacccat

SEQ ID NO: 28 - protein, 19 aa, Homo sapiens
TQHGIRLPLRSGLGGAPLG

SEQ ID NO: 29 - protein, 4 aa, Artificial Sequence
  Synthetic peptide
IETD

SEQ ID NO: 30 - protein, 412 aa, Homo sapiens
DGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILW

SEQ ID NO: 31 - protein, 24 aa, Homo sapiens
TQHGIRLPLRSGLGGAPLGDGLAL

SEQ ID NO: 32 - protein, 770 aa, Homo sapiens
  mat_peptide (18)..(770)
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 33 - protein, 42 aa, Homo sapiens [sequence omitted]

SEQ ID NO: 34 - protein, 4 aa, Artificial Sequence
  Synthetic peptide
HHQK

SEQ ID NO: 35 - protein, 4 aa, Artificial Sequence
  Synthetic peptide
KMDA

SEQ ID NO: 36 - protein, 4 aa, Artificial Sequence
  Synthetic peptide
VFFA

SEQ ID NO: 37 - protein, 4 aa, Artificial Sequence
  Synthetic peptide
FFAE

SEQ ID NO: 38 - protein, 4 aa, Artificial sequence
  Synthetic peptide
NLDA

(19) United States
(12) Patent Application Publication
(10) Pub. No.: US 2009/0081731 A1
(43) Pub. Date: Mar. 26, 2009

(54) Method of producing catalytically active BACE2 enzymes

(76) Inventors: Alfredo Giuseppe Tomasselli, Wildwood, MO (US); Thomas Lowell Emmons, Wentzville, MO (US); Ana Maria Mildner Lallana, Barrio Valle Escondido (AR)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/214,125

(22) Filed: Jun. 17, 2008

Related U.S. Application Data
(63) Continuation of application No. PCT/US2006/048906, filed on Dec. 21, 2006.
(60) Provisional application No. 60/753,430, filed on Dec. 22, 2005.

(51) Int. Cl. C12P 21/06; C12N 9/50

(52) U.S. Cl. 435/69.1; 435/219

(57) ABSTRACT

The invention provides methods and compositions for the efficient expression and isolation of BACE2 polypeptides from inclusion bodies and its refolding to an active enzyme.

### CROSS REFERENCE TO RELATED APPLICATION

This application is a continuation of International Application PCT/US2006/048906, filed Dec. 21, 2006, which claims the benefit of U.S. provisional patent application 60/753,430, filed Dec. 22, 2005.

### FIELD OF THE INVENTION

The invention relates generally to recombinant expression, refolding, and purification of an enzyme. More particularly, the invention relates to methods for preparing purified, active, recombinantly expressed human BACE2.

### BACKGROUND OF THE INVENTION

One of the principal histological hallmarks of Alzheimer's disease (AD) is the presence of senile plaques in the brains of affected patients. AD plaques are composed primarily of amyloid beta or beta amyloid (Aβ) peptides (Selkoe, D., Nature, 399:(6738 Suppl.) A23-31, 1999; Haass et al., Cell, 75:1039-1042,1993; Younkin, S., Ann. Neurol., 37:287-288,1995; and Roher et al. Proc. Natl. Acad. Sci. USA, 90:10836-10840, 1993). These peptides are generated from a large precursor, the β-Amyloid Precursor Protein (APP), by the action of two proteases, referred to as β- and γ-secretases.

β-secretase catalyzes the obligatory first step that liberates the N-terminus of Aβ by cleaving APP between amino acids Met 671 and Asp 672 and between amino acids Tyr 681 and Glu 682 at similar rates (Liu et al., Biochem., 41:3128-3136, 2002; Tomasselli et al., J. Neurosci., 80:1006-10017, 2003), using the numbers of the 770 amino acid isoform (SEQ ID NO: 32) of APP (Asp 672 is also referred to as Asp¹ and Glu 682 as Glu¹¹ to reflect their positions in the Aβ sequence). These two cleavages leave behind membrane-associated APP fragments of 99 and 89 amino acids, respectively, that are subsequently cleaved by γ-secretase at several alternative transmembrane sites to release Aβ peptides of various sizes. Among these fragments, Aβ1-42 (SEQ ID NO: 33) and Aβ11-42 are abundantly found in the plaques of AD patients, and are believed to be very toxic forms of the protein. The toxicity of these peptides is attributed to the presence of the sulfate binding sequence H¹³HQK.

The most prevalent cleavage of APP occurs between Lys 687 (K¹⁶) and Leu 688 (L¹⁷), downstream of the sequence H¹³HQK (SEQ ID NO: 34) by α-secretase protease(s). This latter cleavage generates membrane-associated fragments that are subsequently converted into non-amyloidogenic peptides by the action of γ-secretase.

According to the widely accepted amyloid hypothesis (for a review, see Hardy et al., Science, 297:353-356, 2002), reducing the whole Aβ load may prevent, or slow down, the onset or progression of AD. This hypothesis has attracted interest in finding and targeting the β- and γ-secretases for AD therapy. In spite of intense efforts to identify the γ-secretase, this enzyme has remained elusive, though several reports indicate that a complex of proteins that include presenilin-1 and -2 act as the γ-secretase (for reviews see Fortini, E., Nat. Rev. Mol. Cell Biol., 3:673-684, 2002; Sisodia et al., Nat. Rev. Neurosci., 3:281-290, 2002; Kopan et al., Nat. Rev. Mol. Cell Biol., 5:499-504, 2004).

Efforts to find the β-secretase have resulted in the identification of two membrane-anchored aspartyl proteases, referred to as BACE and BACE2 (Yan et al., Nature, 402:533-537, 1999; Gurney et al., U.S. Pat. No. 6,719,972; Vassar et al., Science, 286:735-741, 1999; Sinha et al., Nature, 402:537-540, 1999; Hussain et al., Mol. Cell Neurosci., 14:419-427, 1999). Although these two enzymes share many similarities, they also have distinct features that may warrant the inhibition of BACE specifically. The enzymes have a 52% amino acid sequence identity (FIG. 1), and both have the structural organization of a classical aspartyl protease consisting of a bi-lobed structure harboring the sequence AspThrGly in one lobe and the sequence AspSerGly on the other lobe. BACE is encoded on chromosome 11q23-24 (Yan et al., Nature, 402:533-537, 1999; Vassar et al., Science, 286:735-741, 1999; Sinha et al., Nature, 402:537-540, 1999; Hussain et al., Mol. Cell Neurosci., 14:419-427, 1999), while BACE2 maps to the Down syndrome critical region of chromosome 21q22.2 (Yan et al., Nature, 402:533-537, 1999; Bennett et al., J. Biol. Chem, 275:20647-20651, 2000; Acquati et al., FEBS Lett., 468:59-64, 2000; Solans et al., Cytogenet. Cell Genet., 89:177-184, 2000).

Both enzymes are synthesized in the endoplasmatic reticulum with a leader sequence and a prosegment that are removed during post-translational modifications. In a classical aspartyl protease, the prosegment portion keeps the enzyme inactive until there is a need for its activity. Upon prosegment removal, auto-catalytically or by another enzyme, full enzymatic activity is released and the enzyme is ready to function. BACE and BACE2 seem to be active with and without prosegment, at least in vitro. Interestingly, in vitro experiments show that BACE2 can auto catalytically remove its prosegment (Yan et al, J. Biol. Chem., 276:36788-36796, 2001; Hussain et al., J. Biol. Chem., 276:23322-23328, 2001) while BACE cannot.

Both BACE and BACE2 are expressed in a variety of tissues including brain, though BACE expression in neurons is much higher than that of BACE2 (Bennett 2000). The two enzymes are anchored to a membrane via a single transmembrane domain and have a cytoplasmic tail. Endogenous BACE is localized essentially to the later Golgi and trans-Golgi network (Vassar et al., Science, 286:735-741, 1999; Yan et al., J. Biol. Chem., 276:34019-34027, 2001; Capell et al., J. Biol. Chem., 275:30849-30854, 2000; Benjiannet et al., J. Biol. Chem., 276:10879-10887, 2001), while BACE2 localizes in the endoplasmatic reticulum, Golgi, trans-Golgi network, endosomes, and plasma membranes (Yan et al., J. Biol. Chem., 276:36788-36796, 2001).

Both recombinantly expressed BACE and BACE purified from natural sources are able to cleave APP at the β-site.

The double mutation Lys670Asn/Met671Leu (Swedish mutation) at the β-site robustly increases the cleavage of APP by both enzymes (BACE and BACE2) at this site (e.g. Yan et al., J. Biol. Chem., 276:36788-36796, 2001 and references cited therein). Notwithstanding, only BACE has been proven to be involved in Aβ production in cells (Yan et al., Nature, 402:533-537, 1999). Specifically, reduction of BACE mRNA by using antisense ribonucleotides in cells resulted in a dramatic decrease in Aβ yields (Yan et al., Nature, 402:533-537, 1999). Transfection of BACE2 plasmid DNA in HEK-293 cells did not increase the β-secretase C99 product, but increased secreted fragments derived from C99 cleavage (Yan et al., J. Biol. Chem., 276:36788-36796, 2001). Indeed, it has been demonstrated that purified BACE2 cleaves the APP between Phe19 and Phe20 of the Aβ1-42 region (SEQ ID NO: 33), and between Phe20 and Ala21 (Yan et al., J. Biol. Chem., 276:36788-36796, 2001). The resulting Phe20 . . . Ala42 and Ala21 . . . Ala42 peptides that do not have the HHQK (SEQ ID NO: 34) motif are not amyloidogenic. Moreover, they are not found in the AD plaques. BACE2 might limit the production of pathogenic forms of Aβ (i.e. fragments beginning at Asp¹ and Glu¹¹). In other words, BACE2 may process APP in a manner such that non-amyloidogenic peptides are generated, as an α-secretase would. Because the potential beneficial function of BACE2 in producing non-amyloidogenic peptides, as well as other unknown cellular roles, it may be advantageous to inhibit BACE specifically, without significant inhibition of BACE2.

Thus, there is a need in the art for compounds, compositions, and methods that inhibit BACE activity without affecting BACE2 activity. Development of such compounds, compositions, and methods are currently hindered by the inability to produce sufficient quantities of active BACE2 necessary for use in drug discovery. Therefore, a simple, efficient, and reliable materials and methods for expression and purification of active recombinant BACE2 is greatly needed. It should also be noted that drugs developed for BACE, but which do not have high selectivity in favor of BACE over BACE2, may be clinically ineffective owing to binding to BACE2 outside the brain, for example, thereby requiring unacceptably high doses.

### SUMMARY OF THE INVENTION

The invention provides materials and methods for the expression, refolding and purification of active recombinant BACE2.

One aspect of the invention provides methods for recombinant protein production, and more specifically for expression, and/or purification, and/or renaturing and/or improving the yield of a recombinant protein, such as an enzyme.

For example, in one variation, the invention is a method for renaturing inactive BACE2 polypeptide into an active BACE2 enzyme comprising: solubilizing an inactive BACE2 polypeptide in an aqueous solution; and incubating the solubilized inactive BACE2 at a temperature of 18° C. to 45° C. until the inactive BACE2 polypeptide folds into an active enzyme. In a preferred variation, the solubilized BACE2 is diluted with several volumes of an aqueous solution, at a colder temperature, prior to the step of incubating at the warmer temperature recited above. For example, between the solubilizing and incubating steps recited above, the method includes an intermediate step of diluting the solubilized inactive BACE2 polypeptide with 20 to 120 volumes of an aqueous solution having a temperature of about 1° C. to 15° C.

For practice of the invention, the BACE2 comprises any BACE2 aspartyl protease amino acid sequence that, when properly folded, exhibits aspartyl protease activity towards a BACE2 substrate as described herein in greater detail. Thus, in a preferred variation, the recombinant BACE2 polypeptide comprises a BACE2 catalytic domain. BACE2, like other aspartyl proteases, comprises a pair of spaced DTG/DSG tripeptide motifs defining the catalytic domain.

One preferred genus of BACE2 amino acid sequences are mammalian sequences, with primates more preferred and human highly preferred.

A human BACE2 sequence is provided in SEQ ID NO: 4, and the invention can be practiced with this full length sequence that includes a signal peptide, propeptide, catalytic region, transmembrane domain, and cytoplasmic tail. As described herein, constructs for prokaryotic expression preferably lack the signal peptide sequence, although a methionine start codon is used. For many subsequent uses, a soluble BACE2 lacking a transmembrane domain (and optionally lacking the cytoplasmic tail too) is preferred. Thus, in one variation, the BACE2 comprises amino acids A41 to W447 of SEQ ID NO:4, or C-terminal truncations thereof that retain BACE2 activity.

Optionally, the inactive BACE2 polypeptide comprises an amino-terminal propeptide. The propeptide is chemically or enzymatically cleavable in a way that does not also cleave (destroy) that portion of BACE2 required for enzymatic activity. In one variation, this propeptide comprises an autocatalysis site for BACE2. In another variation, the amino-terminal cleavable prosegment (propeptide) comprises a caspase cleavage site.

In one preferred variation, the amino-terminal propeptide comprises a portion of a BACE1 peptide fused to a portion of a BACE2 propeptide. In a particular embodiment, the propeptide comprises the amino acid sequence TQHGIRLPLRSGLGGAPLGDGLAL (SEQ ID NO:31).

Additional features and variations of the invention will be apparent to those working in the field of the invention from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each subrange and each value within a range is intended as an aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1 Alignment of BACE (SEQ ID NO: 2) and BACE2 (SEQ ID NO: 4) amino acid sequences. Asterisks identify amino acids that are identical in the two sequences. Boxes indicate the AspThrGly and AspSerGly motifs.

FIG. 2. Conditions explored to refold BACE2 from inclusion bodies (IB).

### DETAILED DESCRIPTION

“BACE2” (beta-site amyloid precursor protein cleaving enzyme 2) is a membrane-bound aspartic protease that is highly homologous with BACE1, both enzymes having a prodomain, a catalytic domain (DTG and DSG), a transmembrane domain, and a short C-terminal cytosolic tail. BACE2 (also known as Asp1 and memapsin 1) has been described, for example, in U.S. Pat. No. 6,706,485 (referred to therein as Asp1) and international patent publication numbers WO 00/17369 and WO 01/23533, which are incorporated herein by reference in their entirety.

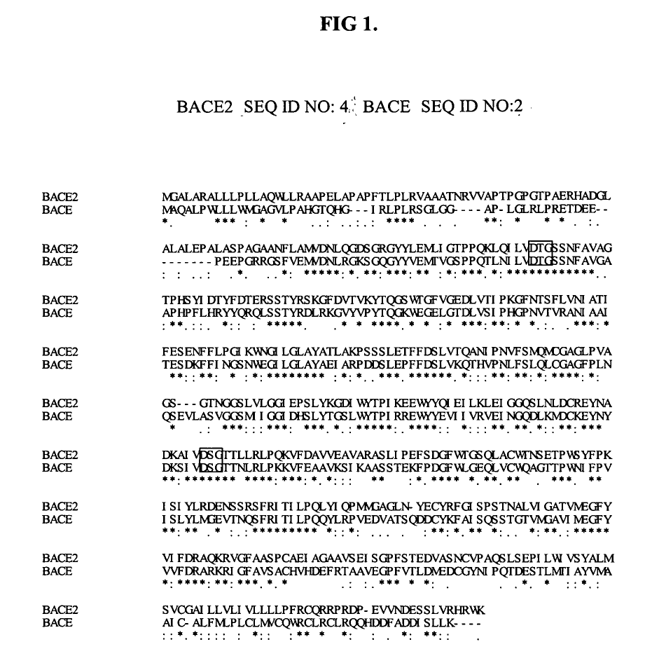

As used herein, “BACE2 polypeptide” refers to full-length BACE2, an active fragment thereof, and fusion proteins comprising full-length BACE2 or an active fragment thereof. An exemplary full-length BACE2 sequence is provided in FIG. 1 (SEQ ID NO: 4). BACE2 polypeptide also includes polypeptides having at least 70%, at least 75%, at least 80%, at least 85%, at least 90%, or at least 95% identity to the sequence set forth in SEQ ID NO: 4, or active fragments thereof. Such polypeptides can contain the addition, substitution, or deletion of one or more amino acids to the polypeptide set forth in SEQ ID NO: 4, or an active fragment thereof. Preferably, the BACE2 polypeptide cleaves a substrate containing the BACE2 cleavage site (e.g., ERHADGLALALEPAWKK (SEQ ID NO:5)) when renatured using the methods of the present invention. Preferably, the BACE2 polypeptide cleaves APP (or APP peptides) at the beta (KM/DA) (SEQ ID NO: 35) or alpha-like secretase sites (VF/FA and FF/AE) (SEQ ID NOS: 36 and 37) recognized by BACE2, within the partial APP sequence of EISEVKMDAEFRHDSGYEVHHQKLVFFAEDVGSNKGAIIGLMVGGVVIATVIVITL (SEQ ID NO: 6). For the purposes of this invention, a polypeptide has BACE2 activity if it cleaves at any of these sites, or at the Swedish mutation (NL/DA) variant site (SEQ ID NO: 38). Preferred polypeptides cleave at least the BACE2 autocatalytic site and an alpha-secretase-like site.

Recombinant BACE2 polypeptide can be produced in a suitable host, such as E. coli, by expressing a construct comprising a nucleic acid sequence encoding the BACE2 polypeptide. The construct can further comprise additional nucleotide sequences that can, for example, assist in the purification and/or expression of the recombinant protein. Furthermore, at least a portion of the nucleic acid sequence encoding the BACE2 polypeptide can be altered to increase translation of the polypeptide. Increased translation can be obtained by altering one or more codons of the sequence to more closely match the codon usage of the suitable host. Codon usage frequencies in a number of hosts are well-known in the art (Nakamura et al., 2000).

When expressed in certain hosts, such as E. coli, recombinant BACE2 polypeptide accumulates in an insoluble form as inclusion bodies. The protein in the inclusion bodies can be a mixture of monomeric and multimeric form of the polypeptide, both reduced and oxidized.

Methods of recovering biologically active, soluble protein from inclusion bodies generally include the steps of: (1) cell lysis, (2) isolation of the inclusion bodies, (3) solubilization of polypeptides from the inclusion bodies, (4) renaturing of the solubilized polypeptides into an active form, and (5) purification of the active polypeptide. Each of these steps are described below in relation to the invention.

Cloning and Expression of BACE2

Expression constructs and methods have been developed for the efficient production, renaturing or refolding, and purification of BACE2 polypeptides. In a preferred embodiment, recombinant BACE2 polypeptide is expressed by a recombinant host, such as a bacterium. The expressed BACE2 polypeptides subsequently are harvested from the recombinant host and refolded or renatured into an active form of the enzyme. The active enzyme can be subjected to additional purification steps. The coding sequences, the expression constructs, the transformed/transfected host cells, the method of expressing the polypeptides, and the methods of refolding and purification are all intended as aspects of the invention.

Useful constructs for the production of BACE2 polypeptide are designed to express a selected portion of the BACE2 polypeptide, for example, express a portion of the BACE2 amino acid sequence shown in FIG. 1. The polynucleotide encoding the BACE2 polypeptide can be operably linked to suitable transcriptional or translational regulatory sequences in an expression construct. Regulatory sequences include transcriptional promoters, operators, enhancers, mRNA ribosomal binding sites, and other sequences that control transcription or translation. Nucleotide sequences are “operably linked” when the regulatory sequence functionally relates to the polynucleotide encoding BACE2 polypeptide. Thus, a promoter nucleotide sequence is operably linked to a polynucleotide encoding BACE2 polypeptide if the promoter nucleotide sequence directs the transcription of the BACE2 polypeptide sequence.

The polynucleotide is cloned into appropriate expression vectors for expression in the appropriate host. Generally, an expression vector will include a selectable marker and an origin of replication, such as for propagation in E. coli. Expression vectors generally comprise one or more phenotypic selectable marker genes. Such genes generally encode, for example, a protein that confers antibiotic resistance or that supplies an auxotrophic requirement.

A polynucleotide can encode a BACE2 polypeptide having an N-terminal methionine (at the beginning of the coding sequence of the expressed polypeptide) to facilitate expression of the recombinant polypeptide in a prokaryotic host, for example, for expression in E. coli. The N-terminal methionine can optionally be cleaved from the expressed BACE2 polypeptide. The polynucleotide can also encode other N-terminal amino acids added to the BACE2 polypeptide that facilitate expression in E. coli. Such amino acids include, but are not limited to, a T7 leader sequence, a T7-caspase 8 leader sequence, and known tags for purification such as the T7-Tag MASMTGGQQMGR [SEQ ID NO:7] that allows binding of antibodies, or a six-histidine tag (His)₆ that allows purification by binding to immobilized metal atom chelate (IMAC) columns, such as those comprised of nickel ions. Other useful peptide tags include the thioredoxin tag, hemaglutinin tag, and GST tag. These and other amino acid tags can be encoded by polynucleotide sequences added to either terminus of the polynucleotide encoding BACE2 polypeptide. In addition, the wild-type polynucleotide sequence expressing human BACE2 can be mutated to provide for codons preferred for the expression of BACE2 polypeptide in E. coli or other desirable host.

The polynucleotide of the expression construct can encode a BACE2 polypeptide that is truncated by removal of all or a portion of the cytoplasmic tail (residues 472 to 496 of SEQ ID NO: 4), the prosequence (residues 1 to 40 of SEQ ID NO: 4, which are typically autocleaved, and further with permissible additional N-terminal cleavages out to about Ala 53), the transmembrane domain (residues 451 to 471 of SEQ ID NO: 4), or any combination of these. The expression constructs can also encode cleavage sites for selected enzymes, such as a caspase, to improve purification of the expressed protein or to assist in expression of the enzyme, when desired.

Examples of suitable constructs for expression in E. coli are presented in Table 1, where E¹ denotes the first encoded amino acid of the prosequence, and encoded amino acids are numbered accordingly. Amino acids depicted in bold are derived from BACE2. Italicized amino acids encoded by Construct 4 indicate the caspase 8 recognition site. pET11a is commercially available from Novagen, Inc., Madison, Wis.; pQE80L is commercially available from Qiagen, Inc., Valencia, Calif. It will be understood that modifications to the specific constructs identified herein can be made within the scope of the invention.

TABLE 1
E. coli-Suitable Constructs Encoding BACE2 polypeptide

| | | |
|---|---|---|
| | | SEQ |
| Construct | Vector/Encoded BACE2 Polypeptide | ID NO: |

| | | |
|---|---|---|
| 1 | pET11a-MASMTGGQQMGRGSM-E¹LAPA . . . LW⁴⁴⁷(H)₆ | 8 |
| 2 | pET11a-MASMTGGQQMGRGS-A⁵³NFL . . . LW⁴⁴⁷(H)₆ | 9 |
| 3 | pQE80L-MRGS(H)₆GS-A⁵³NFL . . . LW⁴⁴⁷ | 10 |
| 4 | pQE80L-MRGS(H)₆GSIETD↓TQHGIRLPLRSGLGGAPLG- | 11 |
| | D³⁶GLAL⁴⁰↓A⁴¹LE . . . LW⁴⁴⁷ | |
| 5 | pQE80L-MRGS(H)₆GS-D³⁶GLAL⁴⁰A⁴¹LE . . . LW⁴⁴⁷ | 12 |

Construct 1 is based on pET11a and encodes a BACE2 polypeptide comprising an N-terminal T7 tag, amino acids E¹-W⁴⁴⁷ of BACE2 (which includes the prosegment, and catalytic domain,), and a C-terminal histidine tag.

Construct 2 is based on pET11a and encodes a BACE2 polypeptide comprising an N-terminal T7 tag, amino acids A⁵³-W⁴⁴⁷ of BACE2 (which lacks 52 amino acids of the prosegment), and a C-terminal histidine tag.

Construct 3 is based on pQE80L and encodes a BACE2 polypeptide comprising an N-terminal histidine tag and amino acids A⁵³-W⁴⁴⁷ of BACE2.

Construct 4 is based on pQE80L and encodes a BACE2 polypeptide comprising an N-terminal histidine tag followed by a caspase-8 cleavage site, amino acids T¹-G¹⁹ of BACE, and amino acids D³⁶-W⁴⁴⁷ of BACE2 (which contains the auto-cleavage site within the prosegment), respectively.

Construct 5 is based on pQE80L and encodes an N-terminal histidine tag and amino acids D³⁶-W⁴⁴⁷ of BACE2.

Each of the above constructs express the BACE2 polypeptide in a lac-inducible manner in E. coli.

Production of BACE2 Polypeptide in E. coli

An expression construct containing a polynucleotide encoding a BACE2 polypeptide can be used to transform bacteria, for example E. coli, in order to produce BACE2 polypeptide. Production of the polypeptide can be inducible or constitutive, depending upon the control elements provided in the vectors. For example, expression constructs are transfected into a bacterial host, such as E. coli BL21 codon plus-RP (Stratagene) or DH5I, and grown in suitable media, such as Luria broth (pH 7.5) supplemented with 50 μg/ml carbenicillin and 34 micrograms/ml chloromphenicol. When cells have grown to a desired density, in general, when the absorbance of the culture at 550 nm is between 0.4 and 0.6, expression is induced. For example, in the expression vectors listed in Table 1, the lac promoter promotes expression of the operably linked BACE2 polynucleotide upon addition of IPTG (for example, to a final concentration of about 1 mM) to the culture media. After induction, for example, about three hours, the cell pellet is collected and can be stored, generally at −70° C., for later inclusion body isolation, enzyme renaturing, and purification.

The expressed recombinant enzyme accumulates intracellularly in an insoluble form, as inclusion bodies. To recover the enzyme from insoluble cellular material, bacterial cells are pelleted from the bacterial cell culture, lysed, and the inclusion bodies are isolated from the lysed cells. The recombinant enzyme can then be isolated from the isolated inclusion bodies.

Generally, lysing of cells to obtain the protein inclusion bodies can be accomplished using a number of known methods, including mechanical and chemical techniques. Sonication and freeze-thaw techniques are generally not practical for the volume of cells being disrupted. However, any commercially available device that uses a pressure differential to disrupt the cells, such as a French Press or a Rannie apparatus, is acceptable, assuming the overall handling capacity is similar or greater than these instruments. Detergent solubilization is not generally a practical solution, since removal of the detergent can pose a difficult challenge and may influence subsequent refolding efforts. Detergents may solubilize contaminating proteins and nucleic acids together with some or all of the protein of interest from the inclusion bodies, and thus is not a desirable option. Once the cells have been lysed, the inclusion bodies may be washed to remove protein contaminants associated with or entrapped in the inclusion bodies.

For example, to obtain inclusion bodies, bacterial cells can be suspended in a suitable buffer that may contain a salt such as sodium chloride, a chelating agent such as EDTA, or both. Suspended cells are then lysed using, for example, a French Press or a Rannie apparatus. The insoluble cellular material obtained is washed in buffer and can be stored and frozen at −20° C.

Protein aggregates (inclusion bodies) are solubilized and then renatured to obtain active protein. Reagents that can be used to solubilize BACE2 polypeptide include denaturants such as urea, guanidine HCl, guanidine thiocyanate, and the like, generally at a concentration of about 6M to 8M. Reducing agents, such as beta-mercaptoethanol (BME), glutathione (gamma-Glu-Cys-Gly; or GSH, Sigma Cat. No. G-6529); or DTT (dithiothreitol, Sigma Cat. No. D-0632), and the like can also be used. These reducing agents can be used separately or in combination to provide the isolated protein in a reduced form (random coil). The reducing agents can reduce the presence of dimers and higher molecular weight multimers, as well as reduce improper folding, for example, as a result of cysteine residues within the protein, or reduce aggregation of the protein.

Solubilization of BACE2 polypeptide present in inclusion bodies can be achieved via treatment with a solubilizing agent (denaturant) at a high pH (about pH 10-11), and in the presence of a reducing agent such as BME. For example, inclusion bodies are extracted with 7.5 M urea, 100 mM BME, 1 mM glycine and 100 mM CAPS (3-[cyclohexylamino]-1-propanesulfonic acid, Sigma Cat. No. C-2632) pH 10.5. After centrifugation, the protein concentration of the supernatant can be adjusted by dilution with a solution of the denaturant to read A₂₈₀ approximately 0.1-4/ml, preferably about 1.2/ml to 1.5/ml at A₂₈₀. Other buffer solutions can be substituted for CAPS, such as AMPSO (3-[(1,1-dimethyl-2-hydroxyethyl)amino]-2-hydroxypropanesulfonic acid, Sigma Cat. No. A1911).

Renaturing BACE2 polypeptide

After the BACE2 polypeptide is solubilized, it is renatured into the correct conformation to provide active enzyme. Typically, renaturing of an expressed recombinant enzyme can be accomplished by removing the solubilizing agent and replacing it with an aqueous buffer, for example, by dilution or dialysis. Generally, renaturing BACE2 polypeptide is accomplished by permitting the diluted enzyme solution to incubate in a cold room (1° C. to abut 15° C.) for about three days to several weeks, followed by incubation at a temperature of 18° C. to 45° C. for about three days to several weeks. Aliquots may be removed during this incubation period to monitor BACE2 activity.

In one aspect of the invention, the solubilized BACE2 polypeptide, prior to dilution, has an absorbance reading of about 0.1 to about 4.0 at 280 nm. Upon dilution of about 10 to about 150 fold, the concentration of the enzyme is expected to be in the range of about 5-50 μg/ml. All discrete concentrations within this range are specifically contemplated, and all subranges of concentration within this range are contemplated for practice of the invention. However, it is expected that other higher or lower concentrations will produce an active protein. For example, it is expected that the BACE2 polypeptide will properly refold in concentrations from about 1 microgram/ml to about 300 micrograms/ml. Thus, the absorbance of the solubilized BACE2 polypeptide solution may be higher or lower, and the solution may be diluted to a greater or lesser extent than 10-150 fold, depending upon the starting concentration (as shown by the absorbance or otherwise) of the solubilized BACE2 polypeptide. The extent of enzyme activity of the renatured protein can be readily determined using the activity assay described herein. Accordingly, for the purposes herein, dilution of the solubilized BACE2 polypeptide refers to the process of diluting a solution of solubilized BACE2 polypeptide to provide a concentration of BACE2 polypeptide capable of refolding to an active enzyme upon incubation in a cold room from a few days to several weeks, followed by incubation at a temperature of 18° C. to 45° C. for about three days to several weeks. Incubation at each discrete temperature within this range is contemplated as an embodiment to practice the invention.

As described in the Examples below, incubation of the diluted BACE2 polypeptide at 18° C. to 45° C. yielded large quantities of active BACE2 enzyme. A number of conditions may be altered from the exemplified conditions and still remain within the scope of the invention. Examples of such conditions include temperature, pH, type and concentration of reducing agent (BME, DTT, GSH), type and concentration of reducing agent pairs (DTT/oxidized DTT; GS/oxidized GS), type and concentration of detergent, type and concentration of co-solvent (glycerol, ethylene glycol), and type and concentration of salt.

Purification of Renatured BACE2 Polypeptide

The renatured enzyme can be purified using standard liquid chromatography techniques, such as, for example, cation or anion exchange chromatography (available, for example, from Amersham Pharmacia Biotech), hydrophobic interaction (available, for example, from Toso Haas), dye interaction (available, for example from Sigma), ceramic hydroxyapatite (available, for example, from Bio-Rad), affinity chromatography (for example, using an inhibitor that binds active enzyme), or size exclusion chromatography (for example, Sephacryl-S 100 or S200 column purification as well as resins from BioRad, Toso Haas, Sigma, and Amersham Pharmacia Biotech). One or a combination of these purification techniques can be used according to the invention to provide purified BACE2 polypeptides. Anion exchange chromatography using, for example, Q-sepharose, Mono-Q, or Resource Q column purification provides useful separation.

In preferred embodiments, the purification procedure comprises three sequential steps: Q-Sepharose, Sephacryl 200, and affinity column. The combination of Q-Sepharose, Sephacryl 200 and affinity columns removes contaminants present in the inclusion bodies. The striking difference between renaturing at 4-8° C. and at 4-8° C. followed by the 37° C. is evident in the elution profile of the Sephacryl columns. When the polypeptides are renatured at 4-8° C. only, the great majority of BACE2 polypeptide is present in inactive multimeric forms. When the polypeptides are renatured at 4-8° C. followed by 37° C., nearly all of the BACE2 polypeptide has the active monomeric form.

In an exemplary embodiment, renatured BACE2 polypeptide is concentrated on an anion exchange column (Q-Sepharose. pH 8.5), dialized at pH 7.2, further concentrated prior to running over a Sephacryl S-200HR column, and fractions therefrom combined according to activity. The combined fractions are dialyzed at pH 8.0 followed by a pH drop to pH 5.0 at 4° C. The BACE2 polypeptide is then subjected to affinity purification using resin containing immobilized inhibitor I-1:

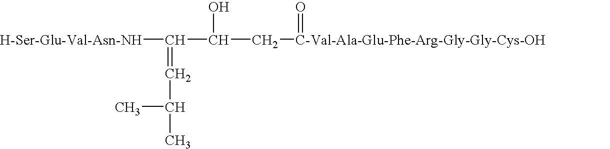

The sample is loaded onto the affinity column at pH 5.0 and eluted at pH 9.5. BACE2 polypeptide obtained from the affinity column can be characterized physically, chemically, or kinetically. In a preferred embodiment, the BACE2 polypeptide is subjected to X-ray crystallography to determine its crystal structure.

It was observed that refolded BACE2 polypeptide is able to trigger its autocleavage during the purification process. The autocleavage between L⁴⁰ and A⁴¹ was essentially complete prior to lowering the pH to 5.0. In large-scale preparations with Protocol II (see Example 4), exposure of BACE2 polypeptide, at relatively low concentrations (0.15 mg/ml) at pH 4.0, at 37° C., triggered cleavage of the enzyme between residues L⁴⁰-A⁴¹, A⁵⁰-G⁵¹, and F⁵⁵-L⁵⁶. In embodiments wherein multiple cleavages is an undesirable event, lowering the pH to 5.0 prior to the affinity column can be carried out at 0-4° C., and the sample then immediately delivered to the affinity column also at 0-4° C. The recovery of the enzyme under these conditions is about 200 mg of BACE2 polypeptide from 16 liters of cell culture.

Activity of Renatured BACE2 Polypeptide

Activity of the renatured, purified BACE2 polypeptide can be determined by incubating the renatured enzyme with a suitable substrate under conditions to allow cleavage of the substrate. The substrate can be labeled with a detectable marker, such as a fluorescent label, to allow detection of cleavage events.

Suitable substrates are peptides that include a BACE2 cleavage site, for example, ERHADGLAL-↓-ALEPAWKK (where ↓ indicates cleavage, SEQ ID NO: 5).

The substrate can be labeled with a suitable detectable marker to permit visualization of cleavage. Assays to detect BACE2 activity can measure retention or liberation of the detectable marker. Suitable detectable markers include, for example, radioactive, enzymatic, chemiluminescent, or fluorescent labels. In some embodiments, the substrate can include internally quenched labels that result in increased detection after cleavage of the substrate. The substrate can be modified to include a paired fluorophore and quencher including, but not limited to, 7-amino-4-methyl coumarin and dinitrophenol, respectively, such that cleavage of the substrate by BACE2 results in increased fluorescence as a result of physical separation of the fluorophore and quencher. Other paired fluorophores and quenchers include bodipy-tetramethylrhodamine and QSY-5 (Molecular Probes, Inc.).

In a variant of this embodiment, biotin or another suitable tag can be placed on one end of the peptide to anchor the peptide to a substrate assay plate, and a fluorophore can be placed at the other end of the peptide. Useful fluorophores include those listed herein, as well as Europium labels such as W8044 (EG&G Wallac, Inc.). One exemplary label is Oregon green that can be coupled to a cysteine residue. Cleavage of the substrate by BACE2 will release the fluorophore or other tag from the plate, allowing detection of an increase in fluorescence signal.

Further examples of detectable markers include a reporter protein amino acid sequence coupled to the substrate. Exemplary reporter proteins include a fluorescing protein (for example, green fluorescing proteins, luciferase, and the like) or an enzyme that is used to cleave a substrate to produce a colorimetric cleavage product. Also contemplated are tag sequences that are commonly used as epitopes for quantitative assays. Preferably, the detectable markers do not interfere with binding of BACE2 to the substrate, or subsequent cleavage of the substrate. For example, detectable markers can be provided in a suitable size that does not interfere with BACE2 activity. In some embodiments, detectable markers can be coupled to the substrate using spacers.

The following examples are included to demonstrate preferred embodiments of the invention. The techniques disclosed in the examples represent techniques that function well in the practice of the invention, and many changes can be made in the specific embodiments and still obtain a similar result without departing from the spirit and scope of the invention.

### EXAMPLES

### Example 1

### Preparation of BACE2 Constructs

This Example describes the preparation of four constructs for expression of BACE2 polypeptides in E. coli. The full length BACE2 cDNA has been previously reported (see, e.g., U.S. Pat. No. 6,706,485 and Yan et al, 2001a). The cDNA and predicted protein sequences of BACE2 described in SEQ ID NOS: 3 and 4 serve as the basis for the nomenclature of the constructs of this Example.

Construct 1

The first 180 nucleotide base pairs of the BACE2 cDNA coding for amino acids E¹ to A⁵⁶ were replaced by a block of synthetic oligonucleotides containing optimized codons for expression in E. coli. Three pairs of oligonucleotides (AMM 605/610, AMM 606/609, and AMM 607/608, Table 2) were annealed and ligated to the NcoI site of vector pQE60 (Qiagen). The introduced changes are indicated in bold type (Table 2). The remaining of the BACE2 cDNA was amplified by PCR using oligos AMM 619 (Forward) and AMM 613 (Reverse). AMM 619 contains an overlap to the newly synthesized cDNA, while AMM 613 introduced a poly histidine at the C-terminus. This PCR product was joined in a new PCR to the synthetic N-terminal portion, resulting in the final insert containing the BACE2 sequence from E¹ to W⁴⁴⁷-(H)₆. This product was digested with BamHI and ligated to vector pET11a (Novagen; Madison, Wis.).

Construct 2

The forward primer (AMM664) introduced a BamHI site immediately preceding the codon for A⁵³. The reverse primer AMM 662 includes pET11a vector sequences and allows amplification including the (H)₆ from construct 1.

Construct 3

The forward primer was AMM 662. The reverse primer AMM663 introduced a HindIII site for cloning into the pQE80 vector, eliminating the (H)₆.

TABLE 2

Oligonucleotides used for Gene Construction
(SEQ ID NOs: are shown below Table)

| | |
|---|---|
| AMM202: | 5′GGCGTATCACGAGGCCCTTTGG |
| | (Foward) |
| AMM605: | 5′CATGGAACTGGCTCCAGCTCCGTTCACTCTGCCACTGCGT |
| | GTTGCTGCAGCTACTAACC |
| | (Forward) |
| AMM606: | 5′GTGTTGTAGCTCCAACTCCAGGACCAGGTACTCCAGCTGA |
| | ACGTCATGCTGATGGTCT |
| | (Forward) |
| AMM607: | 5′GGCTCTGGCTCTGGAACCAGGTCTGGCTCTCCAGCTGGTG |
| | CTGCTAACTTCCTGGC |
| | (Forward) |
| AMM608: | 5′CATGGCCAGGAAGTTAGCAGCACCAGCTGGAGAAGCCAGA |
| | GCTGGTTCCAGAGCCA |
| | (Reverse) |
| AMM609: | 5′GAGCCAGACCATCAGCATGACGTTCAGCTGGAGTACCTGG |
| | TCCTGGAGTTGGAGCTA |
| | (Reverse) |
| AMM610: | 5′CAACACGGTTAGTAGCTGCAGCAACACGCAGTGGCAGAGT |
| | GAACGGAGCTGGAGCCAGTTC |
| | (Reverse) |
| AMM613: | 5′GGAAGCTTAATGGTGATGGTGATGGTGCCAAAAGGGC |
| | (Reverse) |
| AMM619: | 5′CTGCAGGTTGTCTACCATAGCCAGGAAGTTGCAG |
| | (Forward) |
| AMM662: | 5′GGTCAGGATCCAACAGGAGCTCAAGTCAGCT |
| | (Reverse) |
| AMM663: | 5′AAGCTTATCACCACAAAATGGGCTCGCTCAA G |
| | (Reverse) |
| AMM664: | 5′GCTCGGATCCGCTAACTTCCTGGCTATG |
| | (Foward) |
| AMM674: | 5′GCTCGGATCCGATGGTCTGGCTCTGGCTCTG |
| | (Foward) |
| AMM675: | 5′TCCACTGGGTGATGGTCTGGCTCTGGCTCTG |
| | (Foward |
| AMM676: | 5′CCAGACCATCAGGCAGTGGAGCACCACCCAT |
| | (Reverse) |
| AMM202 | (SEQ ID NO: 13) |
| AMM605 | (SEQ ID NO: 14) |
| AMM606 | (SEQ ID NO: 15) |
| AMM607 | (SEQ ID NO: 16) |
| AMM608 | (SEQ ID NO: 17) |
| AMM609 | (SEQ ID NO: 18) |
| AMM610 | (SEQ ID NO: 19) |
| AMM613 | (SEQ ID NO: 20) |
| AMM619 | (SEQ ID NO: 21) |
| AMM662 | (SEQ ID NO: 22) |
| AMM663 | (SEQ ID NO: 23) |
| AMM664 | (SEQ ID NO: 24) |
| AMM674 | (SEQ ID NO: 25) |
| AMM675 | (SEQ ID NO: 26) |
| AMM676 | (SEQ ID NO: 27) |

Construct 4

The N-terminal sequence containing the Caspase8 cleavage site (IETD) and the BACE1 pro-domain from T¹ to G¹⁹ was amplified from pQE80-BACE1, using primers AMM202 and AMM 676. AMM 202 corresponds to pQE80L sequencing forward primer. The AMM 676 reverse primer contains a 10 bp 5′ extension corresponding to the BACE2 sequence D³⁶GLAL. The BACE2 sequence from D³⁶GLAL to W⁴⁴⁷ (PCR2) was amplified using AMM 675 (Forward) and AMM663. The AMM 675 primer contains a 5′ extension corresponding to the APLG¹⁹ BACE1 sequence. Another PCR reaction including the external primers AMM 202 and 663 was used to join PCRs 1 and 2. The final product was digested with BamHI/HindIII and ligated to the corresponding sites of pQE80L. Addition of the BACE1 pro-domain, or fragments thereof, proved very effective in the practice of invention. Additional useful peptide sequences include the full length 24 residue pro-domain of BACE1 (from T¹ to R²⁴), and, in a non-limiting example, all species that include up to about a 10-residue deletion from the N-terminal end of the 24 residue peptide, and all species that include up to about a 10-residue deletion from the C-terminal end of the 24 residue peptide. Additionally, we have observed that the full length BACE2 polypeptide, including the pro-domain (starting at E¹LAPA), can also be effectively refolded using the protocol of the invention identified as Protocol 2.

A description of the protein encoded by each construct is provided in Table 1.

### Example 2

### General Procedures

The PCR conditions were: 5 min at 95° C., followed by 25-30 cycles of 30 sec at 95° C., 30 sec at 60° C. and 2 min at 72° C., with an additional cycle of 5 min at 72° C. PCR products were gel purified prior to restriction digests and ligation reactions. Ligated DNAs were transformed into DH5I cells for propagation and DNA isolations. PCR including 5′ and 3′vector with BACE2 specific primers were performed using the isolated plasmid DNAs as templates. Analysis of these PCR products by agarose gel electrophoresis allowed the selection of clones containing the BACE2 insert in the correct orientation. After the DNA sequence was confirmed on both strands, selected clones were transformed into either E. coli BL21 CodonPlus®-RP (pQE vectors), or E. coli BL21 (DE3) CodonPlus®-RP (PET11a vectors), for expression of the recombinant proteins. The media used was Luria broth supplemented with 100 μg/ml ampicillin.

### Example 3

### BACE2 Polypeptide Expression

For each construct, a single colony containing the recombinant plasmid was amplified in LB (pH 7.5) plus the appropriate antibiotics (50 μg/ml carbenicillin with 34 μg/ml Chloramphenicol) to an absorbance of 0.4-0.5 at 550 nm. Cells were collected by centrifugation, resuspended in LB containing 50 μg/ml carbenicillin and frozen (−70° C.) after the addition of glycerol to a final concentration of 18%.

From the frozen stock, a loop was used to inoculate 100 ml of media, to A₅₅₀=0.02/ml. When the culture reached A₅₅₀=0.5-0.6, expression was induced by addition of IPTG to a final concentration of 1 mM. Expression was allowed to proceed for an additional 3 hours. Cells were collected by centrifugation, weighed, and stored at −70° C. Larger scale cultures were induced with the same method, using 2-liter ferback flasks containing 1 liter of media.

### Example 4

### Inclusion Body Isolation

The E. coli produced recombinant BACE2 polypeptides as inclusion bodies (IBs). Cells from 100 ml cultures were resuspended in 10 ml of TE (10 mM Tris HCl, pH 8.0, 1 mM EDTA) and disrupted by sonication. Cell pellets from larger cultures (1 liter or more) were resuspended with 4-5 ml of TE per liter of culture, and disrupted either in a French Press (16,000 psi) or a Rannie apparatus (12,000 psi). The efficiency of the cell disruption was evaluated by light microscopy. IB were separated from other cellular debris by a series of centrifugation/wash steps, then stored at −70° C.

High expression was observed for all constructs and protein fractions retrieved from inclusion bodies were relatively pure BACE2 polypeptides. The N-terminal sequence of each construct was determined to be authentic by amino acid sequencing of an SDS-PAGE band corresponding to the molecular weight expected for the specific construct analyzed.

### Example 5

### Protein Refolding and Purification

Protocol I. For protein refolding, IBs were dissolved in 7.5M UREA, 100 mM CAPS pH 10.5, 1 mM Glycine, 1 mM EDTA, 100 mM BME (Buffer A). The sample was stirred in this buffer at room temperature for 2-3 h until it became clear. Centrifugation followed at 15000 RPM, Sorval SS34 rotor. The supernatant was recovered and the protein concentration determined by A₂₈₀. Buffer A was added to adjust the protein concentration to read A₂₈₀=1.2-1.5/ml and make the BME 12.5 mM. Various aliquots were taken and diluted 20 to 120 volumes with water at 4-8° C. to determine the effect of dilution on refolding. The various samples were allowed to refold in the cold room, for three weeks. The refolding progress was monitored frequently by the assay described below.

Protocol II. This protocol comprised taking the three-week old refolding mixture generated by Protocol I and allowing it to continue to refold at 37° C. for another three weeks.

### Example 6

### Protein Purification

Q-Sepharose. The refolding mixture was adjusted to 20 mM Tris by adding 1/49 volume of 1M Tris HCl pH 8.0. The pH was adjusted to 8.2-8.5 and loaded onto a Q-sepharose™ Fast Flow column (7-10 ml resin/liter of cell culture) equilibrated with 20 mM Tris HCl, pH 8.2-8.5, 0.2M Urea. The column was washed with 4-6 volumes of the equilibration buffer. BACE2 polypeptide was then eluted with 3-4 volumes of the equilibration buffer containing 0.75M KCl. The sample was then dialyzed at 4° C. against 20 mM HEPES, pH 7.2, 0.2M Urea, with enough potassium chloride to yield a final concentration of 0.2M KCl. Just prior to loading the BACE2 polypeptide onto the SEC column, it was concentrated 20-30 fold using an Amicon stirred cell (Millipore Corp.).

Sephacryl S-200. The concentrated sample from the Q-Sepharose column was delivered to a Sephacryl S-100/200 HR column (2.5×200 cm, and 5×200cm for preparations below and above the 2 liters cell cultures, respectively) equilibrated with 20 mM HEPES, pH 7.2, 0.2M KCl, and 0.2M Urea. Aliquots of 6.5-9 ml were collected in a Gilson FC203B fraction collector, and the A₂₈₀ reading of the fractions were monitored with an ISCO UA-6 UV/VIS absorbance detector. In some cases, the A₂₈₀ reading of the fractions was repeated manually with an UV-601 Spectrophotometer (Shimadzu). Fractions were assayed and combined according to their activity.

Induction of auto cleavage by acid treatment. This step was performed for the samples obtained by refolding at 4-8° C. (Protocol I). The combined fractions from the Sephacryl 200 step were subjected to acidic incubation as follows: one volume of sample was added to 1/10 volume of 1M Na acetate at pH 4.0, 4.5 or 5.0, followed by incubation at 37° C. for 1, 2, 3, 4, 5, 6 hours and overnight to find optimal autocleavage conditions. SDS-PAGE with silver staining was used to analyze the products. The N-terminal sequence of the products was determined after excising the bands from gels blotted onto PDVF (Novex) membrane.

Affinity purification. For BACE2 preparation purposes, this step was done for Protocol II only. However, it was carried out for Protocol I for analytical purposes.

The active post-Sephacryl BACE2 polypeptide fractions were pooled and dialyzed against 10 mM HEPES, pH 8.0. After dialysis, the sample's pH was then lowered to 5.0 with 1 M sodium acetate, 1 M sodium MES, pH 5.0 (0.2 M sodium acetate, 0.2 M sodium MES, final concentration/ea). The sample was then applied to an affinity column using BACE/BACE2 inhibitor I-1 as the ligand (1 mg peptide/1 ml resin). The affinity column was prepared by covalently linking the C-terminal Cys of I-1 to Sulfolink® Coupling Gel which consists of immobilized iodoacetyl groups on a crosslinked agarose support. The procedures and reagents for the coupling were according to the manufacturer. The column was pre-equilibrated with 0.1 M sodium acetate, 0.1 M sodium-MES, pH 5.0. The flow through from the applied sample was recycled through the column two to three times, and the column was washed with six column volumes 0.01 M sodium acetate, 0.01 M sodium-MES, pH 5.0. BACE2 polypeptide was eluted using 3-3.5 column volumes of 0.1 M sodium borate, pH 9.5.

### Example 7

### Activity Assays

The assay reaction, 100-200 μl, contained 0.1 M Sodium acetate pH 4.5, 0.5 to 50 nM of BACE2 polypeptide and 25 or 40 μM of substrate ERHADGLAL-↓-ALEPAWKK (SEQ ID NO: 5) (where ↓ indicates cleavage). The reaction mixture was incubated at 37° C. for two hours, and stopped with ½ volume of 4% TFA. A 25 μL portion of the assay mixture was injected into an Agilent 1100 Series HPLC equipped with a Restek 3μ Allure™ column (4.6 mm i.d., ×30 mm length, C₁₈) pre-equilibrated with 95% reagent A (0.1% TFA in water), 5% reagent B (0.1% TFA in acetonitrile). The flow rate over this column was 1.29 ml per minute. The product and original substrate were eluted from the column using linear gradients (Method I):

| | | |
|---|---|---|
| 0-2.26 minutes | 5-30% | B |
| 2.26-3.40 minutes | 30-50% | B |
| 3.40-3.68 minutes | 50-90% | B |
| 3.68-3.96 minutes | 90-5% | B |
| 3.96-4.53 minutes | 5% | B |

The Agilent 1100 Series HPLC is equipped with a fluorescence detector which allows detection of the substrate disappearance, and ALEPAWKK product formation. Fluorescence emission is monitored at 348 nM upon excitation at 280 nM.

Alternatively, an Alltech Rocket™ column (7 mm i.d., ×53 mm length, C₁₈, 3μ) pre-equilibrated with 88% reagent A (0.1% TFA in water), 12% reagent B (0.1% TFA in acetinitrile) was used. The flow rate over this column was 3 ml per minute. The products were eluted from the column following linear gradients (Method II):

| | | |
|---|---|---|
| 0-4.0 minutes | 12-30% | B |
| 4.0-6.0 minutes | 30-50% | B |
| 6.0-6.5 minutes | 50-90% | B |
| 6.5-7.0 minutes | 90-12% | B |
| 7.0-8.0 minutes | 12% | B |

Peptide detection was as above.

### Example 8

### Active BACE2 Polypeptide Obtained from Recombinant E. coli

BACE2 polypeptide encoded by pET11a-MASMTGGQQMGRGSM-E¹ LAPA . . . LW⁴⁴⁷ (H)₆ (Construct 1, SEQ ID NO: 8) comprises an amino-terminal T7-tag (MASMTGGQQMGR [SEQ ID NO:7]), amino acids 1-447 of BACE2 (SEQ ID NO: 4), and a carboxyl-terminal, six-histidine tag. This BACE2 polypeptide was expressed in E. coli and refolded from inclusion bodies following Protocol 1. It was subjected to Q-Sepharose, Ni-chelate and affinity column with BACE2 inhibitor I-1. The post-Q-Sepharose sample was also analyzed by molecular sieving on Sephacryl 200. The protein was highly pure after Q-Sepharose, but did not bind efficiently to the affinity column, indicating a low level of proper refolding. Analysis of the post-Q-Sepharose column material by Sephacryl 200 revealed two peaks, a major one containing inactive protein and one minor peak containing highly active protein. Acid auto-activation resulted in a complete autoprocessing as established by N-terminus sequencing. The BACE2 that bound to the affinity column was eluted in highly purified form with a high specific activity. Thus, BACE2 could be refolded to an active enzyme from E. coli inclusion bodies.

### Example 9

### Purification of BACE2 Polypeptide Lacking 52 Amino Acids of the Prosegment

Construct 2 (pET11a-MASMTGGQQMGRGS-A⁵³NFL . . . LW⁴⁴⁷(H)₆) (SEQ ID NO: 9) encodes a BACE2 polypeptide comprising an amino-terminal T7 tag, amino acids 53-447 of BACE2 (SEQ ID NO: 4), and a carboxyl-terminal, six-histidine tag. A variety of different buffers, pH's, and concentrations of protein and reducing agents were used to determine optimal refolding conditions. The use of CAPS versus AMPSO buffers at pH from 9.5 to 11.5 for dissolving the inclusion bodies in 7.5 M urea was compared. It was determined that the optimum pH is 10.5 to 11.0, with CAPS yielding better results than AMPSO. Using CAPS at pH 11.0 and diluting the sample with cold water from a protein concentration reading an A₂₈₀=0.5-1.5 to one reading A₂₈₀=0.025 resulted in activity that could be detected after one week, though activity was only detected if the diluted sample was concentrated about 50 times prior to assay. The T7 tag could not be removed from the refolded and purified protein due to lack of the autocleavage site between L⁴⁰ and A⁴¹, and no other cleavage sites were engineered to facilitate the T7 tag removal. The lack of the prosegment containing the auto cleavage site and/or the presence of the T7 tag at the N-terminus of this truncated form of BACE2 could interfere with an efficient refolding.

In an effort to determine whether removal of the T7 tag would facilitate refolding, Construct 3 (pQE80L-MRGS (H)₆ GS-A⁵³NFL . . . SEPI LW⁴⁴⁷, SEQ ID NO: 10) was created. This construct encodes a BACE2 polypeptide comprising an amino-terminal six histidine tag and amino acids 53-447 of BACE2. However, this BACE2 polypeptide showed no activity in spite of a six-week storage in the cold room.

### Example 10

### Purification of BACE2 Polypeptide Comprising D³⁶GLAL to W⁴⁴⁷

BACE2 polypeptide encoded by Construct 4 contains the BACE prosegment from T¹ to G¹⁹ (SEQ ID NO: 28) preceded by a Caspase8 cleavage site, IETD (SEQ ID NO: 29). The BACE prosegment is adjacent to the BACE2 sequence D³⁶GLAL⁴⁰↓A⁴¹LE - - - LW⁴⁴⁷ (SEQ ID NO: 30), which includes the auto cleavage site L⁴⁰↓A⁴¹. Construct 4 was expressed in E. coli to produce BACE2 polypeptide inclusion bodies as described above.

The inclusion bodies were denatured in 7.5 M Urea, 100 mM CAPS, 1 mM glycine, 1 mM EDTA and 100 mM BME and subjected to an extensive exploration of refolding conditions. A solution with a total protein concentration of 0.9 mg/ml in the above buffer at pH 10.6 containing 12.5 mM BME, then diluted 60 fold with water at 4-8° C. gave the best refolding yields. Interestingly, BME at 0.25 mM or less resulted in higher activity and less refolding times than higher concentrations of this reducing agent. The refolding progress was monitored frequently by assaying the activity of the refolding mixture.

Highly pure protein was obtained by a three-step purification. Ion exchange chromatography on Q-Sepharose provided removal of nucleic acids and concentration. This was followed by size exclusion chromatography on Sephacryl 200 which separated the active monomeric BACE2 from the abundant multimeric inactive forms of the enzyme.

The purified protein was analyzed by N-terminus amino acid sequencing. Most of the product (70-80%) contained the intact recombinant protein starting at MRGS (residues 1-4 of SEQ ID NO: 11), while the remaining had undergone autocleavage at the expected site, starting at Ala⁴¹. This means that BACE2 can cleave its prosegment at non-acidic pH.

The yield of purified protein was 0.4 mg/liter of cell culture. The amino acid analysis correlated well with the theoretical composition. Incubation at pH 5.0, 4.5, and 4.0 showed that pH 4.0 resulted in faster and complete autocleavage of the prosegment, and this was removed by dialysis with a membrane cut-off of Mr=10,000-12,000. Inhibitor I-1 covalently attached to a resin retained BACE2 at pH 5.0. The enzyme was first eluted at pH 8.5, but more enzyme came off the column when the pH was raised to pH 9.5. Usually, the affinity step was not carried out for the enzyme prepared in this way as it was already highly pure and completely autocleaved according to SDS-PAGE analysis and to N-terminus sequencing. Moreover the specific activity of recombinant BACE2 did not increase upon the affinity step.

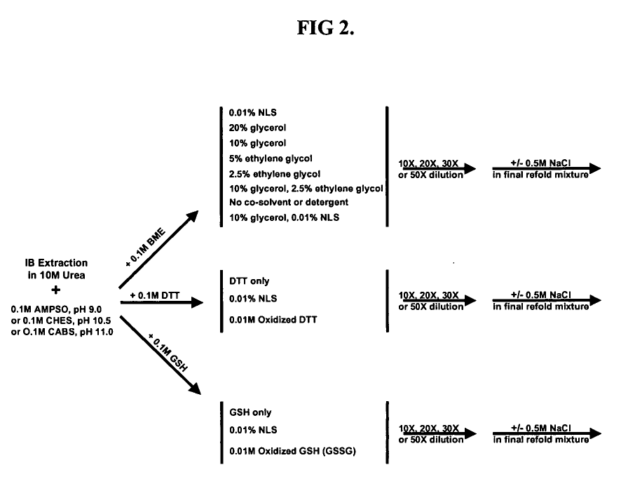

The preparation was scaled up to 8 L cell culture. During the scale up efforts, it was observed that refolding proceeded slowly. In fact, refolding was much slower than that observed in the course of the experiments performed at small-scale. Additional refolding conditions were explored to investigate the role of buffers, pH, salts, dilution, reducing/oxidizing agents, detergents, co-solvents and temperature (FIG. 2). Interestingly, temperature had a striking effect on refolding. Specifically, the activity of a sample that was allowed to refold at room temperature or at 37° C. increased, over time, much faster than the activity of the same sample allowed to renature at 4-8° C.

A set of experiments was carried out to evaluate the effect of the temperature on the rate refolding over time. The best refolding results were obtained if the protein was first allowed to refold at 4-8° C. for three weeks, and then refolding was continued at 37° C. The refolding procedure was perfectly scalable, at least up to 16 liters of cell culture.

Although various specific embodiments of the present invention have been described herein, it is to be understood that the invention is not limited to those precise embodiments and that various changes or modifications can be affected therein by one skilled in the art without departing from the scope and spirit of the invention.

1. A method for renaturing inactive BACE2 polypeptide into an active BACE2 enzyme comprising the steps of:
(a) solubilizing an inactive BACE2 polypeptide in an aqueous solution;
(b) diluting the solubilized inactive BACE2 polypeptide with about 20 to about 2000 volumes of an aqueous solution having a temperature of about 1° C. to 15° C.; and
(c) incubating the diluted solution of step (b) at a temperature of 18° C. to 45° C. until the inactive BACE2 polypeptide folds into an active enzyme.

2. The method of claim 1, wherein the recombinant BACE2 polypeptide comprises a BACE2 catalytic domain.

3. The method of claim 1, wherein the BACE2 comprises amino acids A⁴¹ to W⁴⁴⁷ of SEQ ID NO:4, or C-terminal truncations thereof that retain BACE2 activity.

4. The method of claim 3, wherein the inactive BACE2 polypeptide comprises an amino-terminal propeptide.

5. The method of claim 4, wherein the amino-terminal propeptide comprises a portion of a BACE1 peptide fused to a portion of a BACE2 propeptide.

6. The method of claim 5, wherein the propeptide comprises the amino acid sequence TQHGIRLPLRSGLGGAPLGDGLAL (SEQ ID NO:31).

7. The method of claim 5, wherein the amino-terminal cleavable prosegment comprises a caspase cleavage site.

8. The method of claim 1, wherein said solubilizing comprises dissolving inclusion bodies comprising the inactive BACE2 polypeptide in an aqueous solution containing a denaturant at a pH of about 9.5-11.5 in the presence of a reducing agent.

9. The method of claim 8, wherein the denaturant is urea.

10. The method of claim 8, wherein the reducing agent is β-mercaptoethanol.

11. The method of claim 1, wherein the solubilized inactive BACE2 polypeptide has a protein concentration providing an A₂₈₀ of approximately 0.1-4/ml, preferably an A₂₈₀ of 1.2 to 1.5.

12. The method of claim 11, wherein the solubilized inactive BACE2 polypeptide is diluted 50-60 fold in 4-8° C. water.

13. The method of claim 1, wherein in step (b) thereof, the solubilized inactive BACE2 polypeptide is diluted with about 20 to about 120 volumes of an aqueous solution having a temperature of about 1° C. to 15° C.

14. The method of claim 13, wherein the diluted solubilized inactive BACE2 polypeptide is incubated for at least 7 days at 1° C. to 15° C. prior to step (c).

15. The method of claim 14, wherein the diluted solubilized inactive BACE2 polypeptide is incubated for at least 21 days at 1° C. to 15° C. prior to step (c).

16. The method of claim 1, wherein the diluted inactive BACE2 polypeptide of step (b) is incubated in step (c) for at least two days.

17. The method of claim 16, wherein the diluted inactive BACE2 polypeptide of step (b) is incubated in step (c) for at least seven days.

18. The method of claim 17, wherein the diluted inactive BACE2 polypeptide of step (b) is incubated in step (c) for at least 14 days.

19. The method of claim 18, wherein the diluted inactive BACE2 polypeptide of step (b) is incubated in step (c) for at least twenty-one days.

20. A method of producing active BACE2 polypeptide comprising:
(a) expressing a polynucleotide comprising a nucleotide sequence that encodes a BACE2 polypeptide in a bacteria to produce inclusion bodies that comprise BACE2 polypeptide;
(b) solubilizing the inclusion bodies to solubilize the BACE2 polypeptide therefrom;
(c) reducing the solubilized BACE2 polypeptide with a reducing agent;
(d) diluting the reduced BACE2 polypeptide with an aqueous solution having a temperature of about 1° C. to 15° C.;
(e) incubating the diluted BACE2 polypeptide at a temperature of 1° C. to 15° C.; and
(f) increasing the incubation temperature to 18° C. to 45° C.

21. The method of claim 20, further comprising the step of recovering the active BACE2 polypeptide.

22. The method of claim 20, wherein the bacteria is E. coli.

23. In a method for purifying a recombinant BACE2 polypeptide, the improvement comprising incubating solubilized recombinant BACE2 polypeptide at about 18° C. to 45° C. for at least two days prior to one or more purification steps.

24-51. (canceled)

52. A method of renaturing BACE 2 polypeptide, comprising:
(a) providing a dilute aqueous solution of a BACE2 polypeptide;
(b) incubating the dilute aqueous solution at a temperature of 18° C. to 45° C. for a time effective to renature the BACE2 polypeptide.

53. A method according to claim 52, further comprising a step, prior to said incubating step (b), of maintaining the dilute aqueous solution at a temperature of 0° C. to 15° C. for 1 to 28 days.

54. A method according to claim 52, wherein the providing step comprises:
(a) recombinant expressing the BACE2 polypeptide;
(b) solublizing the BACE2 polypeptide in a denaturant under basic conditions; and
(c) diluting the solublized BACE2 in an aqueous solution to a concentration of between 1 and 300 micrograms/ml, preferably between 5 and 50 mirograms/ml.

55. The method of claim 54, wherein the BACE2 polypeptide comprises a BACE2 catalytic domain.

56. The method of claim 54, wherein the BACE2 comprises amino acids A⁴¹ to W⁴⁴⁷ of SEQ ID NO:4, or C-terminal truncations thereof that retain BACE2 activity.

57. The method of claim 56, wherein the BACE2 polypeptide comprises an amino-terminal propeptide.

58. The method of claim 57, wherein the amino-terminal propeptide comprises a portion of a BACE1 peptide fused to a portion of a BACE2 propeptide.

59. The method of claim 58, wherein the propeptide comprises the amino acid sequence TQHGIRLPLRSGLGGAPLGDGLAL (SEQ ID NO:31).

60. The method of claim 58, wherein the amino-terminal cleavable prosegment comprises a caspase cleavage site.

61. The method of claim 54, wherein said solubilizing comprises dissolving inclusion bodies comprising the BACE2 polypeptide in an aqueous solution containing a denaturant at a pH of about 9.5-11.5 in the presence of a reducing agent.

62. The method of claim 61, wherein the denaturant is urea.

63. The method of claim 61, wherein the reducing agent is β-mercaptoethanol.

64. The method of claim 54, wherein the solubilized BACE2 polypeptide has a protein concentration providing an A₂₈₀ of 1.2 to 1.5.

65. The method of claim 64, wherein the solubilized BACE2 polypeptide is diluted 50-60 fold in 4-8° C. water.

66. The method of claim 54, further comprising the step of incubating the diluted solubilized BACE2 polypeptide for at least 10 days at 1° C. to 15° C. prior to step (c).

67. The method of claim 66, wherein the diluted solubilized BACE2 polypeptide is incubated for at least 7 days at 1° C. to 15° C. prior to step (c).

68. The method of claim 67, wherein the diluted solubilized BACE2 polypeptide is incubated for at least 21 days at 1° C. to 15° C. prior to step (c).

69. The method of claim 54, wherein the diluted BACE2 polypeptide of step (b) is incubated in step (c) for at least two days.

70. The method of claim 69, wherein the diluted BACE2 polypeptide of step (b) is incubated in step (c) for at least seven days.

71. The method of claim 70, wherein the diluted BACE2 polypeptide of step (b) is incubated in step (c) for at least 14 days.

72. The method of claim 71, wherein the diluted BACE2 polypeptide of step (b) is incubated in step (c) for at least twenty-one days.
